(19) United States
(12) Patent Application Publication
(10) Pub. No.: US 2023/0181605 A1
(43) Pub. Date: Jun. 15, 2023

(54) TRIPLE COMBINATION THERAPY FOR ENHANCING CANCER CELL KILLING IN CANCERS WITH LOW IMMUNOGENICITY

(71) Applicant: BeyondSpring Pharmaceuticals, Inc., New York, NY (US)

(72) Inventors: Ramon Mohanlal, New York, NY (US); Lan Huang, New York, NY (US)

(21) Appl. No.: 17/923,189

(22) PCT Filed: Apr. 30, 2021
(86) PCT No.: PCT/US2021/030324

§ 371 Date: Nov. 3, 2022

Related U.S. Application Data
(60) Provisional application No. 63/019,703, filed on May 4, 2020.

(51) Int. Cl. A61K 31/663; A61P 35/00; A61P 25/00; A61K 31/497; A61K 39/395

(52) U.S. Cl. CPC A61K 31/663; A61P 35/00; A61P 25/00; A61K 31/497; A61K 39/3955

(57) ABSTRACT

Disclosed herein are composition and methods of treating a condition where enhanced immunogenicity is desired. Some embodiments disclosed herein relate to compositions comprising a T-cell activator and/or proliferator, one or more immune checkpoint inhibitor, and a FPPS inhibitor. Some embodiments relate to methods of treating cancer by co-administering plinabulin, one or more immune checkpoint inhibitor, and a FPPS inhibitor to a subject in need thereof.

### CROSS REFERENCE TO RELATED APPLICATION

This application is a U.S. National Phase of International Application No. PCT/US2021/030324, filed Apr. 30, 2021, which claims the benefit of priority of U.S. Provisional Application No. 63/019703 filed May 4, 2020, the entire contents of each are herein incorporated by reference.

### BACKGROUND

### Field

The present disclosure relates to the field of chemistry and medicine. More particularly, the present disclosure relates to compositions and methods of treating conditions where low immunogenicity is a rate-limiting factor in achieving an anti-cancer adequate immune response.

### Description of the Related Art

The interaction between malignant cells and the immune system includes elimination of cancer cells by the innate and adaptive immune system, especially by cytotoxic T lymphocytes (CTL) that recognize specific tumor-associated antigens (TAA), or an equilibrium between the immune system and resistant cancer cells, or the evasion of immune control that enables the escape of cancer cells and leads to eventual clinical detection of cancer. Specific immune therapies, such as the cytokine interleukin-2, can drive an existing immune response, and checkpoint inhibitors, such as anti-CTLA-4, and anit-PD-1 and anti-PD-L1, can release an anti-tumor response that was being suppressed by such inhibitory pathways. However, a high percentage of cancer patients lack sufficient immune recognition of their malignant cells that such methods cannot successfully control or eliminate their cancer.

Gliomas are brain tumors originating from glial cells in the nervous system. Two subgroups of gliomas are astrocytomas and oligodendrogliomas. Belonging to the subgroup of astrocytomas, glioblastoma multiforme is the most common malignant brain tumor in adults and accounts for approximately 40% of all malignant brain tumors and approximately 50% of gliomas. it aggressively invades the central nervous system and is ranked at the highest malignancy level (grade IV) among all gliomas. Although there has been steady progress in their treatment due to improvements in neuroimaging, microsurgery, diverse treatment options, such as temozolomide or radiation, g,lioblastomas remain incurable.

Tumor cells of glioblastomas are the most undifferentiated ones among brain tumors, so the tumor cells have high potential of migration and proliferation and are highly invasive, leading to very poor prognosis. Glioblastomas lead to death due to rapid, aggressive, and infiltrative growth in the brain. Glioblastomas are also relatively resistant to radiation and chemotherapy, and, therefore, post-treatment recurrence rates are high. In addition, the immune response to the neoplastic cells is rather ineffective in completely eradicating all neoplastic cells following resection and radiation therapy.

Glioblastoma is classified into primary glioblastoma (de novo) and secondary glioblastoma, depending on differences in the gene mechanism during malignant transformation of undifferentiated astrocytes or glial precursor cells. Secondary glioblastoma occurs in a younger population of up to 45 years of age. During 4 to 5 years, on average, secondary glioblastoma develops from lower-grade astrocytoma through undifferentiated astrocytoma. In contrast, primary glioblastoma predominantly occurs in an older population with a mean age of 55 years. Generally, primary glioblastoma occurs as fu1 minant glioblastoma characterized by tumor progression within 3 months from the state with no clinical or pathological abnormalities.

While cancer remains as an incurable disease for the great majority of patients, in particular in cancers with low immunogenicity, there exists a particular need for developing effective therapeutic agents or regimens that can be used in cancer immunotherapy.

### SUMMARY

Some embodiments relate to a pharmaceutical composition. In some embodiments, the pharmaceutical composition comprises a T-cell activator, one or more immune checkpoint inhibitor, and a Farnesyl Pyrophosphate Synthase (FPPS) inhibitor. In some embodiments, the T-cell activation and/or proliferation is enabled by a tubulin binding agent. In some embodiments, the tubulin binding agent is selected from a group consisting of vinblastine, vincristine, vinorelbine, vinflunine, crytophycin 52, halichondrins, dolastatins, hemiasterlins, colchicine, combretastatins, 2-methyoxyestradiol, E7010, paclitaxel, docetaxel, epothilone, discodermolide, and plinabulin. In some embodiments, the tubulin binding agent is plinabulin.

In some embodiments, the FPPS inhibitor is a nitrogen-containing bisphosphonate compound. In some embodiments, the FPPS inhibitor is quinolone derivative compound or an allosteric non-bisphosphonate compound. In some embodiments, the FPPS inhibitor is selected from pamidronate, alendronate, risedronate, zoledronate, and ibandronat, or an acid or salt thereof.

In some embodiments, the immune checkpoint inhibitor is an inhibitor of PD-1, PD-L1, PD-L2, PD-L3, PD-L4, CTLA-4, LAGS, B7-H3, B7-H4, KIR or TIM3. In some embodiments, the immune checkpoint inhibitor is a PD-1 antibody, a PD-L1 antibody, a PD-L2 antibody, a CTLA-4 antibody, or a combination thereof. In some embodiments, the a PD-1 antibody, a PD-L1 antibody, a PD-L2 antibody, a CTLA-4 antibody is selected from α-CD3-APC, α-CD3-APC-H7, α-CD4-ECD, α-CD4-PB, α-CD8-PE-Cy7, α-CD-8-PerCP-Cy5.5, α-CD11c-APC, α-CD11b-PE-Cy7, α-CD11b-AF700, α-CD14-FITC, α-CD16-PB, α-CD19-AF780, α-CD19-AF700, α-CD2O-PO, α-CD25-PE-Cy7, α-CD40-APC, α-CD45-Biotin, Streptavidin-B V605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137.

In some embodiments, the composition further comprises one or more pharmaceutically acceptable excipients. In some embodiments, the immune checkpoint inhibitor is nivolumab, pembrolizumab, pidilizumab, ipilimumab, BMS 936559, atezolizumab, durvalimumab, or any combinations thereof. In some embodiments, the composition further comprises one or more additional chemotherapeutic agent.

Some embodiments relate to a method for treating cancer. Some embodiments relate to a method for ameliorating cancer in a subject. Some embodiments relate to a method for preventing cancer in a subject. In some embodiments, the method comprises co-administering a T-cell activator, one or more immune checkpoint inhibitor, and a FPPS inhibitor to a subject in need thereof. In some embodiments, the cancer comprises cells expressing farnesyl pyrophosphate synthase.

In some embodiments, the cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, or leukemia. In some embodiments, the cancer is renal cell carcinoma, malignant melanoma, non-small cell lung cancer (NSCLC), ovarian cancer, Hodgkin's lymphoma or squamous cell carcinoma. In some embodiments, the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the cancer is glioblastoma multiforme.

In some embodiments, the method comprises co-administering a first immune checkpoint inhibitor and a second immune checkpoint inhibitor, wherein the first immune checkpoint inhibitor is different from the second immune checkpoint inhibitor. In some embodiments, the first and the second immune checkpoint inhibitor is independently an inhibitor of PD-1, PD-L1, PD-L2, PD-L3, PD-L4, CTLA-4, LAGS, B7-H3, B7-H4, KIR or TIM3. In some embodiments, the first immune checkpoint inhibitor is a PD-1 inhibitor, and the second immune checkpoint inhibitor is a CTLA-4 inhibitor. In some embodiments, the immune checkpoint inhibitor is an antibody. In some embodiments, the immune checkpoint inhibitor is a PD-1 antibody, a PD-L1 antibody, a PD-L2 antibody, or a CTLA-4 antibody.

### BRIEF DESCRIPTION OF THE DRAWINGS

FIG. 1 illustrates various cancer somatic mutation frequency; cancers with low somatic mutation frequency are typically considered to represent low immunogenicity.

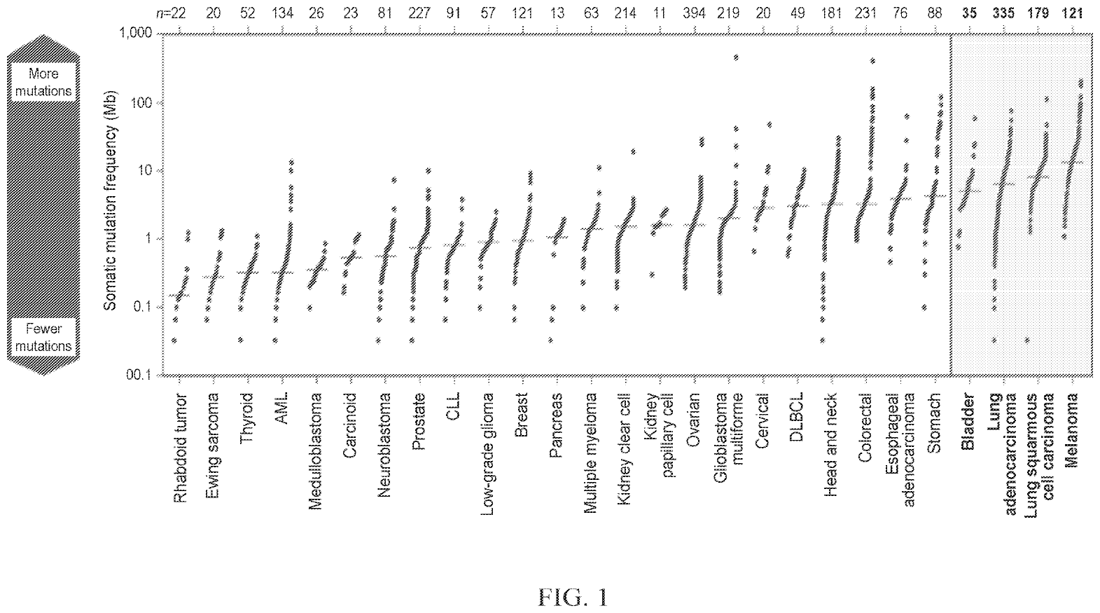

### DETAILED DESCRIPTION

To enable an optimal immune response, it is essential that a foreign antigen is present that is capable of stimulating the immune system (thus antigens that act as immunogens). Many human cancers do not sufficiently induce immunogens, and therefore do not elicit an adequate immune response. These human cancers typically will not be good candidates for immunotherapy (for example PD1-inhibitors, PD-L1 inhibitors, CTLA-4 inhibitors). To overcome non-response to immunotherapy of non- or low-immunogenic cancers, it will be required to covert these into immunogenic cancers, for example by the induction or increased production of neo-antigens that are also immunogenic. In addition, it is important that these neo-antigens/neo-immunogens are optimally processed and presented to effector immune cell by antigen-presenting cells, and that immune checkpoints are adequately inhibited.

An important reason why glioblastoma is resistant to immunotherapy is a relative lack of immunogen induction/production. Glioblastomas are high expressers of the enzyme FDPS (Farnesyl Diphosphate Synthase) (Abate Nature/Scientific Reports 2017), which is also called FPPS (Farnesyl Pyrophosphate Synthase). Therapeutic inhibition of FDPS/FPPS with nitro-bisphosphonates, results in the accumulation of the phosphoantigen, such as isopentenyl pyrophosphate (IPP) which can stimulate T-cells, such as gamma-delta T-Cell. IPP can be converted to the phosphoantigen triphosphoric acid 1-adenosin-5′-yl ester 3-(3-methylbut-3-enyl) ester (Apppl).

In aspects, a therapeutic approach described herein may meet the following criteria: (1) capable of inducing immunogens (antigens that can stimulate the immune system); (2) optimal presentation of these immunogens to effector immune cells that can exert tumor cell-killing; and (3) adequate immune checkpoint inhibition.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. The pharmaceutically acceptable excipient can be a monosaccharide or monosaccharide derivative.

The term “subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or a “therapeutically effective amount” as used herein refers to an amount of a therapeutic agent that is effective to relieve, to some extent, or to reduce the likelihood of onset of, one or more of the symptoms of a disease or condition, and can include curing a disease or condition.

The terms “treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “chemotherapeutic agent” refers to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include but are not limited to, for example, fluoropyrimidines; pyrimidine nucleosides; purine nucleosides; anti-folates, platinum-based agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

The term “ameliorate” as used herein refers to any reduction in the extent, severity, frequency, and/or likelihood of a symptom or clinical sign characteristic of a particular condition.

The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present disclosure may be polyclonal antibodies or monoclonal antibodies. Antibodies also include free antibodies and antigen binding fragments derived therefrom, and conjugates, e.g. pegylated antibodies, drug, radioisotope, or toxin conjugates, and the like. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)). These techniques allow for the screening of particular populations of cells; in immunohistochemistry of biopsy samples; in detecting the presence of markers shed by cancer cells into the blood and other biologic fluids, and the like. Humanized versions of such antibodies are also within the scope of this disclosure. Humanized antibodies are especially useful for in vivo applications in humans due to their low antigenicity.

The terms “cancer”, “neoplasm”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest.

The term “immune checkpoint inhibitor” as used herein refers to a molecule (e.g., small molecule, peptide, polypeptide, protein, antibody, antibody fragment and the like) that acts as an inhibitor (antagonist) of an immune checkpoint pathway. Inhibition of a pathway can include blockade of the pathway through binding to a receptor or signaling molecule that is part of the immune checkpoint pathway.

The term “pharmaceutical carrier”, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. The pharmaceutically acceptable excipient can be a monosaccharide or monosaccharide derivative.

### Compositions

Some embodiments relate to a pharmaceutical composition, comprising a T-cell activator, one or more immune checkpoint inhibitor, and a FPPS inhibitor.

In some embodiments, the T-cell activator is a tubulin binding agent. In some embodiments, the tubulin binding agent is an agent that binds to a Vinca site. In some embodiments, the tubulin binding agent may include vinca alkaloids. In some embodiments, the vinca alkaloid may be selected from vinblastine, vincristine, vinorelbine, vinfluine, dolastatins, cryptophysin, or a combination thereof. In some embodiments, vinca alkyloids are selected from vinblastine, vincristine, and taxanes. In some embodiments, the tubulin binding agent binds near the colchicine binding pocket. In some embodiments, the tubulin binding agent is selected from a group consisting of vinblastine, vincristine, vinorelbine, vinflunine, crytophycin 52, halichondrins, dolastatins, hemiasterlins, colchicine, combretastatins, 2-methyoxyestradiol, E7010, paclitaxel, docetaxel, epothilone, discodermolide, plinabulin, or a combination thereof. In some embodiments, the tubulin binding agent is a taxane. In some embodiments, the tubulin binding agent is docetaxel. In some embodiments, the tubulin binding agent is a combination of plinabulin and a taxane. In some embodiments, the taxane may be selected from paclitaxel, Docetaxel, Cabazitaxel, Larotaxel, Orataxel, Tesetaxel, Milataxel, Taxoprexin, docetaxel-d6-t-Boc, docetaxel-f3-t-Boc, cabazitaxel -7, 10-d6, abeo-taxanel5a.2, BMS-184476, BMS-188797, BMS-275183, SB-T-1214, SB-T-1216, SB-T-12854, SB-T-121602, SB-CST-10202 or DHA-SB-T-1214, or a combination thereof. In some embodiments, the tubulin binding agent is a pharmaceutically acceptable salt of vinblastine, vincristine, vinorelbine, vinflunine, crytophycin 52, halichondrins, dolastatins, hemiasterlins, colchicine, combretastatins, 2-methyoxyestradiol, E7010, paclitaxel, docetaxel, epothilone, discodermolide, plinabulin, or a combination thereof. In some embodiments, the tubulin binding agent is plinabulin.

Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties. In some embodiments, plinabulin can efficiently promote antigen uptake and migration of dendritic cells to lymph nodes where tumor-specific antigens are presented by dendritic cells to prime immune effector cells. Exposure of dendritic cells to plinabulin can induce maturation of dendritic cells and significantly increase their capacity to prime T cells. In some embodiments, plinabulin can mediate tumor size reduction through immune modulation of the tumor microenvironment to promote anti-tumor immune enhancing effects. In some embodiments, substantial therapeutic synergies can be achieved when combining plinabulin with immune checkpoint inhibitors and a farnesyl pyrophosphate synthase (“FPPS”) inhibitor. While not being bound by any particular theory, FPPS inhibitors can promote the generation of phosphoantigens, rendering cells expressing FPPS enzymes (such as glioblastoma cancer cells) more susceptible to attack by the immune system. When combined with T-cell activators (such as plinabulin) and immune checkpoint inhibitors, synergistic immunotherapy effective against cancers such as glioblastomas can be achieved.

In some embodiments, the immune checkpoint inhibitor is an inhibitor of PD-1, PD-L1, PD-L2, PD-L3, PD-L4, CTLA-4, LAGS, B7-H3, B7-H4, KIR or TIM3. A review describing immune checkpoint pathways and the blockade of such pathways with immune checkpoint inhibitor compounds is provided by Pardoll in Nature Reviews Cancer (April, 2012), pages 252-264, which is incorporated herein by reference in its entirety. Immune check point inhibitor compounds display anti-tumor activity by blocking one or more of the endogenous immune checkpoint pathways that downregulate an anti-tumor immune response. The inhibition or blockade of an immune checkpoint pathway typically involves inhibiting a checkpoint receptor and ligand interaction with an immune checkpoint inhibitor compound to reduce or eliminate the down regulation signal and resulting diminishment of the anti-tumor response.

In some embodiments of the present disclosure, the immune checkpoint inhibitor compound inhibits the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, LAG-3, TIM-3, BTLA, and KIR) or by inhibition of a ligand of an immune checkpoint receptor (some examples of ligands include PD-L1 and PD-L2). In such embodiments, the effect of the immune checkpoint inhibitor compound is to reduce or eliminate down regulation of certain aspects of the immune system anti-tumor response in the tumor microenvironment.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8; which are incorporated herein by reference in their entirety). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72, which is incorporated herein by reference in its entirety). Although structurally similar to CTLA-4, PD-1 lacks the MYPPY motif that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43; which are incorporated herein by reference in their entirety). Both PD-L1 and PD-L2 are B7 homologs that bind to PD-1, but do not bind to other CD28 family members. PD-L1 is abundant in a variety of human cancers (Dong et al. (2002) Nat. Med. 8:787-9, which is incorporated herein by reference in its entirety).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745; which are incorporated herein by reference in their entirety). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100; which are incorporated herein by reference in their entirety). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66; which are incorporated herein by reference in their entirety).

The immune checkpoint receptor cytotoxic T-lymphocyte associated antigen 4 (CTLA-4) is expressed on T-cells and is involved in signaling pathways that reduce the level of T-cell activation. It is believed that CTLA-4 can downregulate T-cell activation through competitive binding and sequestration of CD80 and CD86. In addition, CTLA-4 has been shown to be involved in enhancing the immunosuppressive activity of T_{Reg} cells.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

The immune checkpoint receptor T cell membrane protein 3 (TIM-3) is expressed on Th1 and Tc1 cells, but not other T-cells. Interaction of TIM-3 with its ligand, galectin-9, produces a Thl cell death signal. TIM-3 has been reported to play a role in maintaining T-cell exhaustion and blockade of TIM-3 has been shown to restore activity to exhausted T-cells.

The immune checkpoint receptor B- and T-lymphocyte attenuator (BTLA) receptor is expressed on both resting and activated B-cells and T-cells. Activation of BTLA when combined with its ligand HVEM (herpes virus entry mediator) results in downregulation of both T-cell activation and proliferation. HVEM is expressed by certain tumors (e.g., melanoma) and tumor-associated endothelial cells.

The immune checkpoint receptors known as killer cell immunoglobulin-like receptors (KIR) are a polymorphic family of receptors expressed on NK cells and some T-cells and function as regulators of immune tolerance associated with natural killer (NK) cells. Blocking certain KIR receptors with inhibitor compounds can facilitate the destruction of tumors through the increased activity of NK cells.

In some embodiments of the present disclosure, the immune checkpoint inhibitor compound is a small organic molecule (molecular weight less than 1000 daltons), a peptide, a polypeptide, a protein, an antibody, an antibody fragment, or an antibody derivative. In some embodiments, the immune checkpoint inhibitor compound is an antibody. In some embodiments, the antibody is a monoclonal antibody, specifically a human or a humanized monoclonal antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York, which is incorporated herein by reference in its entirety. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081, which are incorporated herein by reference in their entirety. Monoclonal antibodies can be prepared using the general methods described in U.S. Pat. No. 6,331,415 (Cabilly), which is incorporated herein by reference in its entirety.

As an example, human monoclonal antibodies can be prepared using a XenoMouse™ (Abgenix, Freemont, Calif.) or hybridomas of B cells from a XenoMouse. A XenoMouse is a murine host having functional human immunoglobulin genes as described in U.S. Pat. No. 6,162,963 (Kucherlapati), which is incorporated herein by reference in its entirety.

Methods for the preparation and use of immune checkpoint antibodies are described in the following illustrative publications. The preparation and therapeutic uses of anti-CTLA-4 antibodies are described in U.S. Pat. No. 7,229,628 (Allison), U.S. Pat. No. 7,311,910 (Linsley), and U.S. Pat. No. 8,017,144 (Korman), which are incorporated herein by reference in their entirety. The preparation and therapeutic uses of anti-PD-1 antibodies are described in U.S. Pat. No. 8,008,449 (Korman) and U.S. Patent Application No. 2011/0271358 (Freeman), which are incorporated herein by reference in their entirety. The preparation and therapeutic uses of anti-PD-L1 antibodies are described in U.S. Pat. No. 7,943,743 (Korman), which is incorporated herein by reference in its entirety. The preparation and therapeutic uses of anti-TIM-3 antibodies are described in U.S. Pat. No. 8,101,176 (Kuchroo) and U.S. Pat. No. 8,552,156 (Tagayanagi), which are incorporated herein by reference in their entirety. The preparation and therapeutic uses of anti-LAG-3 antibodies are described in U.S. Patent Application No. 2011/0150892 (Thudium) and International Publication Number WO2014/008218 (Lonberg), which are incorporated herein by reference in their entirety. The preparation and therapeutic uses of anti-KIR antibodies are described in U.S. Pat. No. 8,119,775 (Moretta), which is incorporated herein by reference in its entirety. The preparation of antibodies that block BTLA regulated inhibitory pathways (anti-BTLA antibodies) are described in U.S. Pat. No. 8,563,694 (Mataraza), which is incorporated herein by reference in its entirety.

In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a PD-L2 inhibitor or a combined PD-L1/PD-L2 inhibitor. In some embodiments, the immune checkpoint inhibitor is a CTLA-4 inhibitor.

In some embodiments, the composition described herein includes a first immune checkpoint inhibitor and a second immune checkpoint inhibitor, wherein the first immune checkpoint inhibitor is different from the second immune checkpoint inhibitor. In some embodiments, the first and the second immune checkpoint inhibitor is independently an inhibitor of PD-1, PD-L1, PD-L2, PD-L3, PD-L4, CTLA-4, LAG3, B7-H3, B7-H4, KIR or TIM3. In some embodiments, the first immune checkpoint inhibitor is a PD-1 inhibitor, and the second immune checkpoint inhibitor is a CTLA-4 inhibitor. In some embodiments, the first immune checkpoint inhibitor is a PD-L1 inhibitor, and the second immune checkpoint inhibitor is a CTLA-4 inhibitor. In some embodiments, the first immune checkpoint inhibitor is a PD-L2 inhibitor, and the second immune checkpoint inhibitor is a CTLA-4 inhibitor.

In some embodiments, the immune checkpoint inhibitor can be a small peptide agent that can inhibit T cell regulation function. In some embodiments, the immune checkpoint inhibitor can be a small molecule (e.g. less than 500 Daltons) that can inhibit T cell regulation function. In some embodiments, the immune checkpoint inhibitor can be a molecule providing co-stimulation of T-cell activation. In some embodiments, the immune checkpoint inhibitor can be a molecule providing co-stimulation of natural killer cell, CD8 T-cell, or CD4 T-cell activation. In some embodiments, the immune checkpoint inhibitor can be an antibody. In some embodiments, the immune checkpoint inhibitor is a PD-1 antibody. In some embodiments, the immune checkpoint inhibitor is a PD-L1 antibody. In some embodiments, the immune checkpoint inhibitor is a PD-L2 antibody. In some embodiments, the immune checkpoint inhibitor is a PD-L3 antibody. In some embodiments, the immune checkpoint inhibitor is a PD-L4 antibody. In some embodiments, the immune checkpoint inhibitor is a CTLA-4 antibody. In some embodiments, the immune checkpoint inhibitor is an antibody binding to CTLA-4, LAG3, B7-H3, B7-H4, KIR, or TIM3.

In some embodiments, the immune checkpoint inhibitor is pembrolizumab, nivolumab, cemiplimab, atezolizumab, avelumab, pembrolizumab, pidilizumab, ipilimumab, BMS 936559, durvalumab, or any combinations thereof. In some embodiments, the one or more immune checkpoint inhibitor may include an anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449, which is incorporated herein by reference in its entirety. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743, which is incorporated herein by reference in its entirety.

The antibody can be selected from α-CD3-APC, α-CD3-APC-H7, α-CD4-ECD, α-CD4-PB, α-CD8-PE-Cy7, a-CD-8-PerCP-Cy5.5, α-CD1 1c-APC, α-CD1 1b-PE-Cy7, α-CD1 1b-AF700, α-CD14-FITC, α-CD16-PB, α-CD19-AF780, α-CD19-AF700, α-CD20-PO, α-CD25-PE-Cy7, α-CD40-APC, α-CD45-Biotin, Streptavidin-BV605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137.

A variety of antibodies (Abs) can be used in the composition described herein, including antibodies having high-affinity binding to PD-1 PD-Ll, PD-L2, PD-L3, or PD-L4. Human mAbs (HuMAbs) that bind specifically to PD-1 (e.g., bind to human PD-1 and may cross-react with PD-1 from other species, such as cynomolgus monkey) with high affinity have been disclosed in U.S. Patent No. 8,008,449, which is incorporated herein by reference in its entirety. HuMAbs that bind specifically to PD-Ll with high affinity have been disclosed in U.S. Patent No. 7,943,743, which is incorporated herein by reference in its entirety. Other anti-PD-1 mAbs have been described in, for example, U.S. Pat. Nos. 6,808,710, 7,488,802 and 8,168,757, and PCT Publication No. WO 2012/145493, all of which are incorporated herein by reference in their entireties. Anti-PD-Ll mAbs have been described in, for example, U.S. Pat. Nos. 7,635,757 and 8,217,149, U.S. Publication No. 2009/0317368, and PCT Publication Nos. WO 2011/066389 and WO 2012/14549, all of which are incorporated herein by reference in their entireties.

In some embodiments, the anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743.

In some embodiments, the FPPS inhibitor generates phosphoantigens. In some embodiments, the FPPS inhibitor selectively inhibits an FPPS enzyme. In some embodiments, the FPPS inhibitor selectively inhibits an FPPS enzyme associated with glioblastoma. In some embodiments, the FPPS inhibitor may selectively inhibit one or more of an FPPS, GGPPS, DDPPS, DHDDS, and FDPS (Farnesyl Diphosphate Synthase) enzyme. In some embodiments, the FPPS inhibitor selectively inhibits an FPPS enzyme in a cell containing said enzyme or a cancer cell containing said enzyme with an FPPS inhibitor, wherein the FPPS inhibitor is capable of selectively inhibiting the FPPS enzyme. In some embodiments, the cancer cell is glioblastoma. In some embodiments, the FPPS inhibitor causes the glioblastoma to become more immunogenic.

In some embodiments, the FPPS inhibitor is a nitrogen-containing bisphosphonate compound. In some embodiments, the FPPS inhibitor is a quinoline derivative compound. In some embodiments, the FPPS inhibitor is an allosteric non-bisphosphonate compound.

In some embodiments, the FPPS inhibitor is selected from pamidronate (Aredia®), alendronate (Fosamax®), risedronate (Actonel®), zoledronate (Zometa®), and ibandronate (Boniva®), neridronate, risedronate, minodronate, TH-Z93, TH-Z97, and their salts and acids.

In some embodiments, the FPPS inhibitor is selected from one or more of the following:

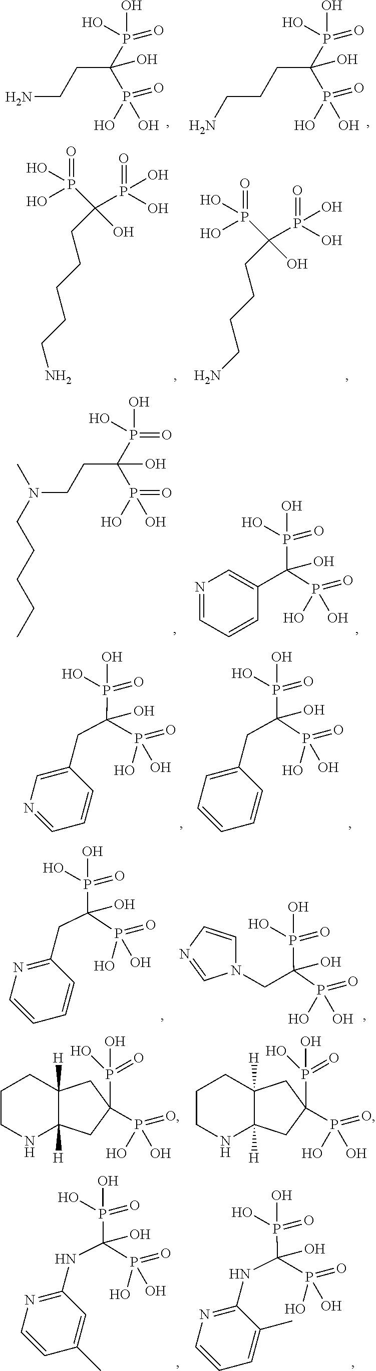

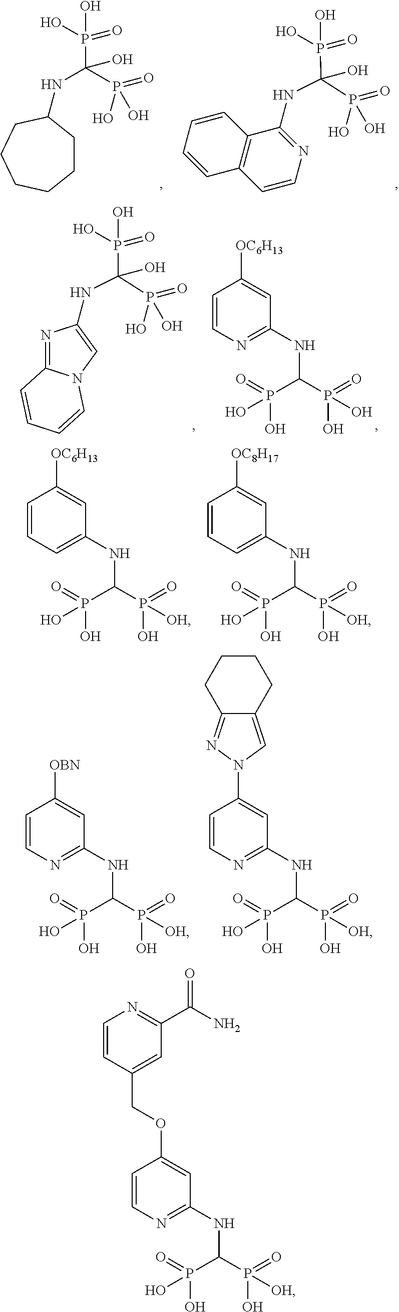

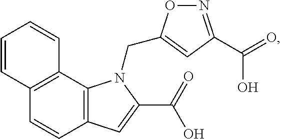 [SMILES: O=C(O)c1cc(Cn2c(C(=O)O)cc3ccc4ccccc4c32)on1]

or a pharmaceutically acceptable salt thereof.

In some embodiments, the composition can further include one or more pharmaceutically acceptable diluents. In some embodiments, the pharmaceutically acceptable diluent can include Kolliphor HS15® (Polyoxyl (15)-hydroxystearate). In some embodiments, the pharmaceutically acceptable diluent can include propylene glycol. In some embodiments, the pharmaceutically acceptable diluents can include kolliphor and propylene glycol. In some embodiments, the pharmaceutically acceptable diluents can include kolliphor and propylene glycol, wherein the kolliphor is about 40% by weight and propylene glycol is about 60% by weight based on the total weight of the diluents. In some embodiments, the composition can further include one or more other pharmaceutically acceptable excipients.

Standard pharmaceutical formulation techniques can be used to make the pharmaceutical compositions described herein, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of plinabulin or pharmaceutically acceptable salts thereof; (b) an immune checkpoint inhibitor; (c) a FPPS inhibitor; and (d) a pharmaceutically acceptable carrier, diluent, excipient or combination thereof.

Other embodiments include co-administering plinabulin, one or more immune checkpoint inhibitors, and a FPPS inhibitor in separate compositions. Thus, some embodiments include a first pharmaceutical compositions comprising: (a) a safe and therapeutically effective amount of plinabulin or pharmaceutically acceptable salts thereof and a pharmaceutically acceptable carrier, diluent, excipient or combination thereof; (b) a second pharmaceutical composition comprising one or more immune checkpoint inhibitor and a pharmaceutically acceptable carrier, diluent, excipient or combination thereof; and (c) a third pharmaceutical composition comprising a FPPS inhibitor or pharmaceutically acceptable salts thereof and a pharmaceutically acceptable carrier, diluent, excipient or combination thereof.

### Method of Treatment

Some embodiments relate to a method for treating cancer using the pharmaceutical composition described herein to a subject in need thereof. Some embodiments relate to a method for treating cancer, comprising co-administering a T-cell activator, one or more immune checkpoint inhibitor, and a FPPS inhibitor to a subject in need thereof. In some embodiments, the subject can be an animal, e.g., a mammal, a human. In some embodiments, the subject is a human.

Some embodiments relate to methods of providing co-stimulation of T-cell activation against cancer by co-administering a T-cell activator, one or more immune checkpoint inhibitor, and a FPPS inhibitor. Some embodiments relate to methods of providing co-stimulation of natural killer cells against cancer by co-administering a T-cell activator, one or more immune checkpoint inhibitor, and a FPPS inhibitor.

In some embodiments, the therapy described herein may treat a bone resorption disease. In some embodiments, the bone resorption disease is selected from the group consisting of osteoporosis, hypercalcemia due to malignancy, and Paget' s disease. In some embodiments, the therapy described herein may target a FPPS in osteoclasts. In some embodiments, the therapy described herein may activate gamma delta T-cells, CD8 T-cells, or CD T-cells to kill tumor cells. In some embodiments, the gamma delta T-cells contain a Vγ2 Vδ2 T-cell receptor.

In some embodiments, the cancer comprises cancer cells expressing farnesyl pyrophosphate synthetase. In some embodiments, the cancer cells expressing farnesyl pyrophosphate synthetase comprises leukemia, kidney cancer, liver cancer, adrenal carcinoma, bladder cancer, mammary cancer, stomach cancer, gastric tumor cancer, ovarian cancer, colorectal carcinoma, the rectum cancer, prostate cancer, carcinoma of the pancreas, lung cancer, carcinoma of vagina or thyroid carcinoma, sarcoma, glioblastoma multiforme, multiple myeloma or gastrointestinal cancer, colorectal carcinoma, colorectal adenomas, neck tumors, head tumor, tumorigenesis, tumorigenesis, myelomatosis, myelodysplastic syndrome, AML (acute myeloid leukemia), AMM (agnogenic myeloid metaplasia (angiogenic myeloid metaplasia)), mesothelioma, neurospongioma, or osteocarcinoma. In some embodiments, the cancer cells expressing farnesyl pyrophosphate synthetase is glioblastoma multiforme.

In some embodiments, the cancer comprises cancer cells expressing a binding ligand of PD-1. In some embodiments, the binding ligand of PD-1 is PD-L1. In some embodiments, the binding ligand of PD-1 is PD-L2.

In some embodiments, the method of treating cancer described herein further includes identifying cancer cells expressing a binding ligand of PD-1. In some embodiments, the method of ameliorating cancer in a subject described herein further includes identifying cancer cells expressing a binding ligand of PD-1. In some embodiments, the method of treating cancer described herein further includes identifying cancer cells expressing PD-L1. In some embodiments, the method of treating cancer described herein further includes identifying cancer cells expressing PD-L2. In some embodiments, the method of treating cancer described herein further includes identifying cancer cells expressing PD-L3 or PD-L4.

In some embodiments, identifying cancer cells expressing a binding ligand of PD-1 includes using an assay to detect the presence of the binding ligand. Examples of applicable assay include but are not limited to PD-L1 IHC 22C3 pharmDx kit and PD-L1 IHC 28-8 pharmDx available from Dako. In some embodiments, identifying cancer cells with FPPS expression includes using a FPPS diagnostic based on IHC, gene expression-based assay or other relevant assay.

In some embodiments, the cancer comprises cancer cells expressing a binding ligand of CTLA-4. In some embodiments, the binding ligand of CTLA-4 is B7.1 or B7.2.

In some embodiments, the method of treating, ameliorating, or preventing cancer in a subject described herein further includes identifying cancer cells expressing a binding ligand of CTLA-4. In some embodiments, the method of treating cancer described herein further includes identifying cancer cells expressing B7.1 or B7.2.

In some embodiments, the one or more immune checkpoint inhibitor may be incorporated in a pharmaceutically acceptable formulation. In some embodiments, the one or more immune checkpoint inhibitor is incorporated in a pharmaceutically acceptable aqueous formulation. Examples of acceptable aqueous formulations include isotonic buffered and pH 4.5-8 adjusted saline solutions such as Lactated Ringer's Solution and the like.

In some embodiments, the immune checkpoint inhibitor compound is incorporated in a pharmaceutically acceptable liposome formulation, wherein the formulation is a passive or targeted liposome formulation. Examples of methods for the preparation of suitable liposome formulations of antibodies are described U.S. Pat. No. 5,399,331 (Loughrey), U.S. Pat. No. 8,304,565 (Wu) and U.S. Pat. No. 7,780,882 (Chang), which are incorporated herein by reference in their entirety.

In some embodiments, the immune checkpoint inhibitor is pembrolizumab, nivolumab, cemiplimab, atezolizumab, avelumab, pembrolizumab, pidilizumab, ipilimumab, BMS 936559, durvalumab, or any combinations thereof. In some embodiments, the one or more immune checkpoint inhibitor may include an anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449, which is incorporated herein by reference in its entirety. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743, which is incorporated herein by reference in its entirety.

In some embodiments, the cancer may be a cancer that is usually treated with with one of the following therapy: a chemotherapy, a radiotherapy, an hormonotherapy, an immunotherapy, a specific kinase inhibitor-based therapy, an antiangiogenic agent based-therapy, an antibody-based therapy and a surgery. In some embodiments, the cancer may be selected from a breast cancer, a prostate cancer, an oesophagus cancer, a colon cancer, a rectal cancer, a kidney cancer, a lung cancer, in particular a non-small cell lung cancer (NSCLC), a thyroid cancer, an osteosarcoma, a gastrointestinal sarcoma (GIST), a melanoma, a leukaemia, in particular an acute lymphoid leukemia, an Hodgkin lymphoma, and a neuroblastoma.

In some embodiments, the cancer is a low-grade immunogenic cancer.

In some embodiments, the cancer is rhabdoid tumor, Ewing sarcoma, thyroid cancer, acute myeloid leukemia (AML), medulloblastoma cancer, carcinoid cancer, neuroblastoma, prostate cancer, chronic lymphocytic leukemia (CLL), low-grade glioma, breast cancer, pancreas, multiple myeloma, kidney papillary cell, ovarian cancer, glioblastoma multiforme, cervical, diffuse large B-cell lymphoma (DLBCL), head and neck, colorectal, esophageal adenocarcinoma, bladder cancer, lung adenosacrinoma, lung squamous cell carcinoma, or melanoma. In some embodiments, the cancer is bladder cancer, lung adenocarcinoma, lung squamous cell carcinoma, or melanoma.

In some embodiments, the cancer is lung cancer, stomach cancer, colon cancer, pancreatic cancer, prostate cancer, kidney cancer, bladder cancer, ovary cancer, cervical cancer, glioblastoma, myeloma, lymphoma, or leukemia. In some embodiments, the cancer is renal cell carcinoma, malignant melanoma, non-small cell lung cancer (NSCLC), ovarian cancer, Hodgkin's lymphoma or squamous cell carcinoma. In some embodiments, the cancer is selected from breast cancer, colon cancer, rectal cancer, lung cancer, prostate cancer, melanoma, leukemia, ovarian cancer, gastric cancer, renal cell carcinoma, and liver cancer. In some embodiments, the cancer is a solid tumor or hematological cancer.

In some embodiments, the cancer does not have any cells expressing PD-1, PD-L1, or PD-L2 at detectable levels.

Some embodiments relate to a method of disrupting cancer associated tumor vasculature in a subject comprising co-administering to the subject a compound of plinabulin, one or more immune checkpoint inhibitor, and a FPPS inhibitor.

Various cancers are associated the formation of tumor vasculature. In some embodiments, the cancer is selected from the group consisting of a melanoma, a pancreatic cancer, a colorectal adenocarcinoma, a brain tumor, acute lymphoblastic leukemia, chronic lymphocytic leukemia, hormone refractory metastatic prostate cancer, metastatic breast cancer, non-small cell lung cancer, renal cell carcinoma, head and neck cancer, prostate cancer, colon cancer, anaplastic thyroid cancer.

In some embodiments, if any component of the triple combination does not penetrate the Blood Brain Barrier (BBB), a device may be used to enhance BBB penetration. In some embodiments, the device is a device producing ultrasound capable of making the BBB more permeable. In some embodiments, an additional therapeutic agent capable of enhancing BBB penetration is also provided. In some embodiments, the additional therapeutic agent is a drug with nanoparticles capable of enhancing BBB penetration.

Some embodiments include co-administering a composition, and/or pharmaceutical composition described herein, with an additional medicament. For example, as described above, some embodiments include co-administering of a tubulin binding agent with one or more immune checkpoint inhibitor, and a FPPS inhibitor.

In some embodiments, a method for halting or reversing a progressive cancer in a subject comprising co-administering a tubulin binding agent, a FPPS inhibitor, with one or more additional chemotherapeutic agents, one or more immune checkpoint inhibitors, and/or radiation, as described above. In some embodiments, the method includes co-administering a tubulin binding agent, one or more immune checkpoint inhibitor, a FPPS inhibitor and radiation. In some embodiments, the tubulin binding agent is plinabulin. In some embodiments, the one or more immune checkpoint inhibitor includes a PD-1, PD-L1, PD-L2, or a CTLA-4 inhibitor.

In some embodiments, the progressive cancer may be selected from breast cancer, bladder cancer, glioblastoma, glioblastoma multiforme, metastatic brain tumor, head and neck cancer, non-small cell lung cancer, small cell lung cancer, colorectal cancer, gastrointestinal cancer, gastroesophageal cancer, renal cell cancer, prostate cancer, liver cancer, colon cancer, pancreatic cancer tumor, ovarian cancer tumor, lymphoma, cutaneous T-cell lymphoma, sarcoma, multiple myeloma, or melanoma.

In some embodiments, the present disclosure provides a method for treating a solid tumor. In some embodiments, the present disclosure provides a method for ameliorating a solid tumor. In some embodiments, the present disclosure provides a method for preventing a solid tumor. In some embodiments, the method may include administering a tubulin binding agent, one or more immune checkpoint inhibitor, and a FPPS inhibitor. In some embodiments, the present disclosure provides a method for treating a breast cancer tumor, a bladder cancer tumor, a glioblastoma tumor, metastatic brain tumor, a head and neck cancer tumor, a non-small cell lung cancer tumor, a small cell lung cancer tumor, a colorectal cancer tumor, a gastrointestinal stromal tumor, a gastroesophageal carcinoma, a renal cell cancer tumor, a prostate cancer tumor, a liver cancer tumor, a colon cancer tumor, a pancreatic cancer tumor, an ovarian cancer tumor, a lymphoma, a cutaneous T-cell lymphoma, a sarcoma, a multiple myeloma, or a melanoma. In some embodiments, the present disclosure provides a method for treating an immune suppressed tumor. An immune suppressed tumor is a tumor that contains immune suppressive associated cells such as for example T_{Reg} cells, myeloid derived suppressor cells (MDSC), M2 macrophages, and the like or immune suppressive factors such as inducible nitric oxide synthase (iNOS), PD-L1, and the like.

In some embodiments, the cancer comprises cancer cells expressing a binding ligand of PD-1. In some embodiments, the binding ligand of PD-1 is PD-Ll. In some embodiments, the binding ligand of PD-1 is PD-L2.

In some embodiments, the method of treating cancer described herein further includes identifying cancer cells expressing a binding ligand of PD-1. In some embodiments, the method of treating cancer described herein further includes identifying cancer cells expressing PD-L1. In some embodiments, the method of treating cancer described herein further includes identifying cancer cells expressing PD-L2.

In some embodiments, identifying cancer cells expressing a binding ligand of PD-1 includes using an assay to detect the presence of the binding ligand. Examples of applicable assay include but are not limited to PD-L1 IHC 22C3 pharmDx kit and PD-L1 IHC 28-8 pharmDx available from Dako.

In some embodiments, the cancer comprises cancer cells expressing a binding ligand of CTLA-4. In some embodiments, the binding ligand of CTLA-4 is B7.1 or B7.2.

In some embodiments, the method of treating cancer described herein further includes identifying cancer cells expressing a binding ligand of CTLA-4. In some embodiments, the method of treating cancer described herein further includes identifying cancer cells expressing B7.1 or B7.2.

In some embodiments, cancer is head and neck cancer, lung cancer, stomach cancer, colon cancer, pancreatic cancer, prostate cancer, breast cancer, kidney cancer, bladder cancer, ovary cancer, cervical cancer, melanoma, gliomas including glioblastoma, myeloma, lymphoma, sarcoma, multiple myeloma, or leukemia. In some embodiments, the cancer is renal cell carcinoma, malignant melanoma, non-small cell lung cancer (NSCLC), ovarian cancer, Hodgkin's lymphoma or squamous cell carcinoma. In some embodiments, the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the cancer is a solid tumor or hematological cancer.

In some embodiments, the cancer does not have any cells expressing PD-1, PD-L1, or PD-L2 at detectable levels.

In some embodiments, the cancer is selected from breast cancer, colon cancer, glioma, metastatic brain tumor, rectal cancer, lung cancer, prostate cancer, melanoma, leukemia, ovarian cancer, gastric cancer, renal cell carcinoma, liver cancer, pancreatic cancer, lymphomas, sarcoma, multiple myeloma, and myeloma. In some embodiments, the cancer is a solid tumor or hematological cancer.

In some embodiments, the subject can be an animal, e.g., a mammal, a human. In some embodiments, the subject is a human.

In some embodiments, the formulations or compositions described herein are incorporated in a pharmaceutically acceptable solution. In some embodiments, the formulations or compositions described herein are incorporated in an injectable formulation. In some embodiments, the formulations or compositions described herein are incorporated in an injectable formulation that substantially maintains the formulations or compositions described herein at or near the injection site.

The precise amount of the composition described herein incorporated in a particular method or therapeutic combination of the disclosure may vary according to factors known in art such as for example, the physical and clinical status of the subject, the method of administration, the content of the formulation, the intended dosing regimen or sequence.

In some embodiments, the treatment cycle can include co-administering plinabulin, one or more immune checkpoint inhibitor, and a FPPS inhibitor in combination with administering plinabulin alone, administering one or more checkpoint inhibitor alone, or administering a FPPS inhibitor alone. In some embodiments, plinabulin and one or more immune checkpoint inhibitor are co-administered on day 1, followed by administration of plinabulin alone after 1 day, 2 days, 3 days, 4 days, 5 days, 6 days, 7 days, 2 weeks, or 3 weeks, and then followed by co-administration of plinabulin, one or more immune checkpoint inhibitor, and a FPPS inhibitor after 1 day, 2 days, 3 days, 4 days, 5 days, 6 days, 7 days, 2 weeks, or 3 weeks. In some embodiments, plinabulin, one or more immune checkpoint inhibitor, and a FPPS inhibitor are administered simultaneously on day 1, followed by administration of plinabulin or one or more immune checkpoint inhibitor alone on a day selected between day 2 and day 31, and then followed by co-administration of plinabulin, one or more immune checkpoint inhibitor, and a FPPS inhibitor on a day selected between day 3 and day 31. In some embodiments, plinabulin, one or more immune checkpoint inhibitor, and a FPPS inhibitor are co-administered on day 1, followed by administration of plinabulin alone on day 8, and then followed by co-administration of plinabulin, one or more immune checkpoint inhibitor, and a FPPS inhibitor on day 15. In some embodiments, the treatment cycle can be repeated two or more times.

In some embodiments, the composition or pharmaceutical compositions described herein may further comprise an additional chemotherapeutic agent. In some embodiments, an additional chemotherapeutic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, \ Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi , Avastin (Bevacizumab), Axitinib, Azacitidine, BEACOPP, Becenum (Carmustine), B eleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Cabazitaxel, Cabozantinib-S-Malate, CAF, Campath (Alemtuzumab), Camptosar (Irinotecan Hydrochloride), Capecitabine, CAPDX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine) , Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN ,Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate),Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idelalisib, Ifex (Ifosfamide), Ifosfamide, IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Tressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate) ,Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Margibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP,Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neupogen (Filgrastim), Nexavar (Sorafenib Tosylate) , Nilotinib, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV , Pegaspargase, Peginterferon Alfa-2b, PEG-Intron (Peginterferon Alfa-2b), Pembrolizumab, Pemetrexed Disodium Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc),Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thioguanine, Thiotepa, Tolak (Fluorouracil-Topical), Toposar (Etoposide), Topotecan Hydrochloride, Toremifene, Torisel (Temsirolimus), Tositumomab and Iodine I 131, Tositumomab, Totect (Dexrazoxane Hydrochloride), TPF, Trabectedin, Trametinib, Trastuzumab, Treanda (Bendamustine Hydrochloride) , Trifluridine and Tipiracil Hydrochloride, Trisenox (Arsenic Trioxide), Tykerb (Lap atinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI,XELOX, Xgeva (Denosumab), Xofigo (Radium 223 Dichloride), Xtandi (Enzalutamide), Yervoy (Ipilimumab),Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

### Radiation Therapy

In some embodiments, the composition as described herein may be co-administered with radiation. In some embodiments, the radiation may be selected from external beam radiation therapy or internal radiation therapy. In some embodiments, the external beam radiation therapy may be selected from three-dimensional conformal radiation therapy (3D-CRT), intensity modulated radiation therapy (IMRT), proton beam therapy, image-guided radiation therapy (IGRT), Stereotactic radiation therapy (SRT), or a combination thereof. In some embodiments, the radiation may be selected from intraoperative radiation therapy (IORT), systemic radiation therapy, radioimmunotherapy, radiosensitizers, radioprotectors, or a combination thereof.

### Administration

Administration of the pharmaceutical compositions described herein can be via any of the accepted modes of administration for agents that serve similar utilities including, but not limited to, orally, sublingually, buccally, subcutaneously, intravenously, intranasally, topically, transdermally, intradermally, intraperitoneally, intramuscularly, intrapulmonarilly, vaginally, rectally, or intraocularly. Oral and parenteral administrations are customary in treating the indications that are the subject of the preferred embodiments.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 min utes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In some embodiments, the one or more immune checkpoint inhibitor may be an antibody. In some embodiments, the antibody is a dry, lyophilized solid that is reconstituted with an aqueous reconstitution solvent prior to use. In some embodiments, the antibody is incorporated in a pharmaceutically acceptable formulation and the pharmaceutically acceptable formulation is injected directly into a tumor. In some embodiments, the immune checkpoint inhibitor antibody is incorporated in a pharmaceutically acceptable formulation and the pharmaceutically acceptable formulation is injected into the peritumoral region surrounding a tumor. The peritumoral region may contain antitumor immune cells. In some embodiments, the antibody is incorporated in a pharmaceutically acceptable formulation and the pharmaceutically acceptable formulation is administered by intravenous injection or infusion. In some embodiments, the immune checkpoint inhibitor antibody is incorporated in a pharmaceutically acceptable formulation and the pharmaceutically acceptable formulation is administered by subcutaneous injection or intradermal injection. In some embodiments, the antibody is incorporated in a pharmaceutically acceptable formulation and the pharmaceutically acceptable formulation is administered by intraperitoneal injection or lavage.

The precise amount of immune checkpoint inhibitor compound incorporated in a particular method or therapeutic combination of the disclosure may vary according to factors known in art such as for example, the physical and clinical status of the subject, the method of administration, the content of the formulation, the physical and chemical nature of the immune checkpoint inhibitor compound, the intended dosing regimen or sequence. Those of ordinary skill in the art, however, can readily determine the appropriate amount with due consideration of such factors.

Various oral dosage forms can be used, including such solid forms as tablets, capsules (e.g. solid gel capsules and liquid gel capsules), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include additional drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a daily dose of Plinabulin may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. In some embodiments, a daily dose of an immune checkpoint inhibitor may be from about 0.5 mg/kg to about 320 mg/kg or more of body weight, from about 0.5 mg/kg or less to about 240 mg/kg, from about 1.0 mg/kg to about 120 mg/kg of body weight, or from about 3 mg/kg to about 50 mg/kg of body weight. In some embodiments, a daily dose of a FPPS inhibitor may be from about 3 mg to about 150 mg per dose, from about 5 mg or less to about 100 mg, from about 10 mg to about 75 mg per dose, or from about 35 mg to about 50 mg per dose.

In some embodiments, tubulin binding agent may be administered at a dose in the range of about 1 mg/m² to about 50 mg/m². In some embodiments, the tubulin binding agent is administered at a dose in the range of about 1-50 mg/m² of the body surface area. In some embodiments, the tubulin binding agent is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-34.5, 27.5-32.5, 2-20, 2.5-22.5, or 9.5-21.5 mg/m², of the body surface area. In some embodiments, the tubulin binding agent is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the tubulin binding agent is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, vis administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the tubulin binding agent dose is about 5 mg-300 mg, 5 mg-200 mg, 7.5 mg-200 mg, 10 mg-100 mg, 15 mg-100 mg, 20 mg-100 mg, 30 mg- 100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the tubulin binding agent administered is about 20 mg-60 mg, 27 mg-60 mg, 20 mg-45 mg, or 27 mg-45 mg. In some embodiments, the tubulin binding agent administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the tubulin binding agent dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the tubulin binding agent dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

In some embodiments, a dose of one or more immune checkpoint inhibitors may be from about 100 μg to about 1000 mg, from about 500 μg or less to about 800 mg, from about 1.0 mg to about 600 mg, from about 100 mg to about 600 mg, or from about 200 mg to 500 mg. In some embodiments, a dose of one or more immune checkpoint inhibitors may be from about 240 mg to about 480 mg per dose. In some embodiments, the dose of the one or more immune checkpoint inhibitors is about 240 mg. In some embodiments, the dose of the one or more immune checkpoint inhibitors is about 480 mg.

In some embodiments, one or more immune checkpoint inhibitors may be administered at a dose in the range of about 100 mg/kg to about 5000 mg/kg. In some embodiments, one or more immune checkpoint inhibitors is administered at a dose in the range of about 100-1000 mg/kg. In some embodiments, one or more immune checkpoint inhibitors is administered at a dose in the range of about 100-200, 100-300, 100-400, 100-500, 100-600, 100-700, 100-800, 100-900, 100-1000, 100-1100, 100-1200, 100-1300, 100-1375, 100-1400, 100-1500, 100-1600, 100-1700, 100-1800, 100-1900, 100-2000, 100-2250, 100-2500, 100-2750, 100-3000, 150-200, 150-300, 150-400, 150-500, 150-600, 150-700, 150-800, 150-900, 150-1000, 150-1100, 150-1200, 150-1300, 150-1375, 150-1400, 150-1500, 150-1600, 150-1700, 150-1800, 150-1900, 150-2000, 150-2250, 150-2500, 150-2750, 150-3000, 250-2000, 250-3000, 250-4000, 250-5000, 250-600, 250-700, 250-800, 250-900, 250-1000, 250-1100, 250-1200, 250-1300, 250-1375, 250-1400, 250-1500, 250-1600, 250-1700, 250-1800, 250-1900, 250-2000, 250-2250, 250-2500, 250-2750, 250-3000, 250-750, 300-400, 300-500, 300-600, 300-700, 300-800, 300-900, 300-1000, 300-1100, 300-1200, 300-1300, 300-1375, 300-1400, 300-1500, 300-1600, 300-1700, 300-1800, 300-1900, 300-2000, 300-2250, 300-2500, 300-2750, or 300-3000, mg/kg. In some embodiments, one or more immune checkpoint inhibitors is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 45, 50, 55, 60, 65, 70, 75, 80, 85, 90, 95, or 100 mg.

In some embodiments, one or more immune checkpoint inhibitor dose is about 0.5 mg-3000 mg, 0.5 mg-2500 mg, 0.5 mg-2000 mg, 0.5 mg-1500 mg, 0.5 mg-1000 mg, 0.5 mg-500 mg,0.5 mg-200 mg, 0.75 mg-200 mg, 1.0 mg-100 mg, 1.5 mg-100 mg, 2.0 mg-100 mg, 3.0 mg-100 mg, 4.0 mg-100 mg, 1.0 mg-80 mg, 1.5 mg-80 mg, 2.0 mg-80 mg, 3.0 mg-80 mg, 4.0 mg-80 mg, 1.0 mg-60 mg, 1.5 mg-60 mg, 2.0 mg-60 mg, 3.0 mg-60 mg, or about 4.0 mg-60 mg. In some embodiments, one or more immune checkpoint inhibitors administered is about 20 mg-60 mg, 27 mg-60 mg, 20 mg-45 mg, or 27 mg-45 mg. In some embodiments, one or more immune checkpoint inhibitors administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, 52 mg-60 mg, 100 mg-200 mg, 200 mg-300 mg, 300 mg-400 mg, 400 mg-500 mg, 500 mg-1000 mg, 1000 mg-2000 mg, or 1000 mg-3000 mg . In some embodiments, one or more immune checkpoint inhibitor dose is greater than about 1 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, one or more immune checkpoint inhibitor dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg, about 300 mg, about 400 mg, about 500 mg, about 1000 mg, about 2000 mg, or about 3000 mg.

In some embodiments, a dose of one or more FPPS inhibitors may be from about 100 ᵢ.t.g to about 1000 mg, from about 500 ᵢ.t.g or less to about 800 mg, from about 1.0 mg to about 600 mg, from about 100 mg to about 600 mg, or from about 200 mg to 500 mg. In some embodiments, a dose of one or more FPPS inhibitors may be from about 240 mg to about 480 mg per dose. In some embodiments, the dose of the one or more FPPS inhibitors is about 240 mg. In some embodiments, the dose of the one or more FPPS inhibitors is about 480 mg.

In some embodiments, one or more FPPS inhibitors may be administered at a dose in the range of about 100 mg/kg to about 5000 mg/kg. In some embodiments, one or more FPPS inhibitors is administered at a dose in the range of about 100-1000 mg/kg. In some embodiments, one or more FPPS inhibitors is administered at a dose in the range of about 100-200, 100-300, 100-400, 100-500, 100-600, 100-700, 100-800, 100-900, 100-1000, 100-1100, 100-1200, 100-1300, 100-1375, 100-1400, 100-1500, 100-1600, 100-1700, 100-1800, 100-1900, 100-2000, 100-2250, 100-2500, 100-2750, 100-3000, 150-200, 150-300, 150-400, 150-500, 150-600, 150-700, 150-800, 150-900, 150-1000, 150-1100, 150-1200, 150-1300, 150-1375, 150-1400, 150-1500, 150-1600, 150-1700, 150-1800, 150-1900, 150-2000, 150-2250, 150-2500, 150-2750, 150-3000, 250-2000, 250-3000, 250-4000, 250-5000, 250-600, 250-700, 250-800, 250-900, 250-1000, 250-1100, 250-1200, 250-1300, 250-1375, 250-1400, 250-1500, 250-1600, 250-1700, 250-1800, 250-1900, 250-2000, 250-2250, 250-2500, 250-2750, 250-3000, 250-750, 300-400, 300-500, 300-600, 300-700, 300-800, 300-900, 300-1000, 300-1100, 300-1200, 300-1300, 300-1375, 300-1400, 300-1500, 300-1600, 300-1700, 300-1800, 300-1900, 300-2000, 300-2250, 300-2500, 300-2750, or 300-3000, mg/kg. In some embodiments, one or more FPPS inhibitors is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 45, 50, 55, 60, 65, 70, 75, 80, 85, 90, 95, or 100 mg.

In some embodiments, one or more FPPS inhibitor dose is about 0.5 mg-3000 mg, 0.5 mg-2500 mg, 0.5 mg-2000 mg, 0.5 mg-1500 mg, 0.5 mg-1000 mg, 0.5 mg-500 mg,0.5 mg-200 mg, 0.75 mg-200 mg, 1.0 mg-100 mg, 1.5 mg-100 mg, 2.0 mg - 100 mg, 3.0 mg-100 mg, 4.0 mg-100 mg, 1.0 mg-80 mg, 1.5 mg-80 mg, 2.0 mg-80 mg, 3.0 mg-80 mg, 4.0 mg-80 mg, 1.0 mg-60 mg, 1.5 mg-60 mg, 2.0 mg-60 mg, 3.0 mg-60 mg, or about 4.0 mg-60 mg. In some embodiments, one or more FPPS inhibitors administered is about 20 mg-60 mg, 27 mg-60 mg, 20 mg-45 mg, or 27 mg-45 mg. In some embodiments, one or more FPPS inhibitors administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, 52 mg-60 mg, 100 mg-200 mg, 200 mg-300 mg, 300 mg-400 mg, 400 mg-500 mg, 500 mg-1000 mg, 1000 mg-2000 mg, or 1000 mg-3000 mg. In some embodiments, one or more FPPS inhibitor dose is greater than about 1 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, one or more FPPS inhibitor dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg, about 300 mg, about 400 mg, about 500 mg, about 1000 mg, about 2000 mg, or about 3000 mg.

In some embodiments, the initial dose of one or more immune checkpoint inhibitor is 1 mg on day 1 followed a dose of a second immune checkpoint inhibitor is 3 mg.

In some embodiments, the tubulin binding agent is administered prior to the administration of one or more immune checkpoint inhibitor. In some embodiments, the tubulin binding agent is administered concurrently with one or more immune checkpoint inhibitor. In some embodiments, the tubulin binding agent is administered after one or more immune checkpoint inhibitor. In some embodiments, the tubulin binding agent is administered prior to the administration of one or more immune checkpoint inhibitor and the FPPS inhibitor. In some embodiments, the tubulin binding agent is administered after the administration of one or more immune checkpoint inhibitor and the FPPS inhibitor.

In some embodiments, the tubulin binding agent is administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 24 h, 30 h, 36 h, 40 h, or 48 h after the administration of one or more immune checkpoint inhibitor or the FPPS inhibitor. In some embodiments, the tubulin binding agent is administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 24 h, 30 h, 36 h, 40 h, or 48 h before the administration of one or more immune checkpoint inhibitor or the FPPS inhibitor. In some embodiments, the tubulin binding agent is administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 30 h, 36 h, 40 h, or 48 h after the administration of one or more immune checkpoint inhibitor or the FPPS inhibitor. In some embodiments, the tubulin binding agent is administered in more than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h30 h, 36 h, 40 h, or 48 h after the administration of one or more immune checkpoint inhibitor or the FPPS inhibitor. In some embodiments, the tubulin binding agent is administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 30 h, 36 h, 40 h, or 48 h after the administration of one or more immune checkpoint inhibitor. In some embodiments, the tubulin binding agent is administered in more than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h30 h, 36 h, 40 h, or 48 h before the administration of one or more immune checkpoint inhibitor. In some embodiments, the tubulin binding agent is administered in about 1 min-5 min, 1 min-10 min, 1 min-15 min, 1 min-20 min, 1 min -25 min, 1 min -30 min, 0.25 h-0.5 h, 0.25-0.75 h, 0.25-1 h,0.5 h-1 h, 0.5 h-2 h, 0.5 h-2.5 h, 1 h-2 h, 1 h-3 h, 1 h-5 h, 1 h-24 h, 1 min-24 h, or 1 min -2 h, 1 day- 2days, 1 day-3days, 1 day-4 days, 1 day-5 days, or 1 day-6 days after the administration of one or more immune checkpoint inhibitor. In some embodiments, the tubulin binding agent is administered in about 1 min-5 min, 1 min-10 min, 1 min-15 min, 1 min-20 min, 1 min -25 min, 1 min -30 min, 0.25 h-0.5 h, 0.25-0.75 h, 0.25-1 h,0.5 h-1 h, 0.5 h-2 h, 0.5 h-2.5 h, 1 h-2 h, 1 h-3 h, 1 h-5 h, 1 h-24 h, 1 min-24 h, or 1 min -2 h, 1 day- 2days, 1 day-3days, 1 day-4 days, 1 day-5 days, or 1 day-6 before the administration of one or more immune checkpoint inhibitor.

In some embodiments, the tubulin binding agent, the one or more immune checkpoint inhibitor, and the FPPS inhibitor are co-administered. As used herein, the terms “co-administer,” “co-administering,” or “co-administration,” refers to two or more agents or therapies that have a biological effect on a subject at the same time, regardless of when or how they are actually administered. In one embodiment, the agents or therapies are administered simultaneously. In one such embodiment, administration in combination is accomplished by combining the agents in a single dosage form. In another embodiment, the agents or therapies are administered sequentially. In some embodiments, the administration may be separated by a period of time, for example, 30 min utes, 1 hour, 2 hours, 1 day, 2 days, 3 days, or 1 week. In one embodiment the agents are administered through the same route, such as orally. In another embodiment, the agents are administered through different routes, such as one being administered orally and another being administered i.v.

In some embodiments where an immune checkpoint inhibitor is co-administered with a tubulin binding agent and a FPPS inhibitor, a method for treating a subject having a cancer or tumor may include administering a therapeutically effective amount of a tubulin binding agent, or a pharmaceutically acceptable salt thereof, after the subject is administered the one or more immune checkpoint inhibitor and a FPPS inhibitor. In some embodiments, a method of inhibiting the growth of cancer or tumor cells in a subject may include administering a therapeutically effective amount of a tubulin binding agent, or a pharmaceutically acceptable salt thereof, after the subject is administered one or more immune checkpoint inhibitor and a FPPS inhibitor. In some embodiments, a method for increasing a cell-mediated immune response of a cell population may include administering a therapeutically effective amount of a tubulin binding agent, or a pharmaceutically acceptable salt thereof, after administering one or more immune checkpoint inhibitor and a FPPS inhibitor.

In some embodiments, a tubulin binding agent is co-administered with a CTLA-4 receptor inhibitor and a FPPS inhibitor. In some embodiments, a tubulin binding agent may be co-administered with a PD-1 or PD-L1 receptor inhibitor compound and a FPPS inhibitor.

In some embodiments, the method comprises treating a subject by co-administering a therapeutically effective amount of a tubulin binding agent, a FPPS inhibitor, and a LAG-3 receptor inhibitor compound. In some embodiments, the method comprises treating a subject by co-administering a therapeutically effective amount of a tubulin binding agent, a FPPS inhibitor, and a TIM-3 receptor inhibitor compound. In some embodiments, the method comprises treating a subject by co-administering a therapeutically effective amount of a tubulin binding agent, a FPPS inhibitor, and a BTLA receptor inhibitor compound. In some embodiments, the method comprises treating a subject by co-administering a therapeutically effective amount of a tubulin binding agent, a FPPS inhibitor, and a KIR receptor inhibitor compound. In some embodiments, the method comprises treating a subject by co-administering a therapeutically effective amount of a tubulin binding agent, a FPPS inhibitor, and a PD-L1 inhibitor compound. In some embodiments, the method comprises treating a subject by co-administering a therapeutically effective amount of a tubulin binding agent, a FPPS inhibitor, and a PD-L2 inhibitor compound.

In some embodiments of the present disclosure, the method comprises treating a subject by co-administering a therapeutically effective amount of a tubulin binding agent, a FPPS inhibitor, and a blocking antibody of an immune checkpoint pathway. In some embodiments, the method comprises treating a subject by co-administering a therapeutically effective amount of a tubulin binding agent, a FPPS inhibitor, and an anti-CTLA-4 receptor antibody. In some embodiments, the method comprises treating a subject by co-administering a therapeutically effective amount of a tubulin binding agent, a FPPS inhibitor, and an anti-PD-1 receptor antibody.

In some embodiments, the method comprises co-administering to a subject having a tumor a therapeutically effective amount of a tubulin binding agent, a FPPS inhibitor, and an anti-LAG-3 receptor antibody. In some embodiments, the method comprises co-administering to a subject having a tumor a therapeutically effective amount of a tubulin binding agent, a FPPS inhibitor, and an anti-TIM-3 receptor antibody. In some embodiments, the method comprises co-administering to a subject having a tumor a therapeutically effective amount of a tubulin binding agent, a FPPS inhibitor, and an anti-BTLA receptor antibody. In some embodiments, the method comprises co-administering to a subject having a tumor a therapeutically effective amount of a tubulin binding agent, a FPPS inhibitor, and an anti-KIR receptor antibody. In some embodiments, the anti-KIR receptor antibody is lirilumab. In some embodiments, the method comprises co-administering to a subject having a tumor a therapeutically effective amount of a tubulin binding agent, a FPPS inhibitor, and an anti-PD-1 antibody. In some embodiments, the anti-PD-1 antibody is lambrolizumab, pidilizumab, or nivolumab. In some embodiments, the method comprises co-administering to a subject having a tumor a therapeutically effective amount of a tubulin binding agent, a FPPS inhibitor, and an anti-PD-L1 antibody. In some embodiments, the method comprises co-administering to a subject having a tumor a therapeutically effective amount of t a tubulin binding agent, a FPPS inhibitor, and an anti-PD-L2 antibody. In some embodiments, the method comprises co-administering to a subject having a tumor a therapeutically effective amount of a tubulin binding agent, a FPPS inhibitor, and an anti-CTLA-4 antibody. In some embodiments, the anti-CTLA-4 antibody is ipilimumab or tremelimumab.

In some embodiments, when the tubulin binding agent is administered prior to one or more immune checkpoint inhibitor administration, the tubulin binding agent is administered about 1 min-5 min, 1 min- 10 min, 1 min-15 min, 1 min-20 min, 1 min -25 min, 1 min-30 min, 0.25 h-0.5 h, 0.25-0.75 h, 0.25-1 h,0.5 h-1h, 0.5 h-2 h, 0.5 h-2.5 h, 1 h-2 h, 1 h-3 h, 1 h-5 h, 1 h-24 h, 1 min-1 h, 1 min-2 h, 1 min-5 h, 1 min-24 h, 1 day- 2days, 1 day-3days, 1 day-4 days, 1 day-5 days, or 1 day-6 days before the administration of the one or more immune checkpoint inhibitor. In some embodiments, the tubulin binding agent is administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 30 h, 36 h, 40 h, 48 h, 4 days, 5 days, 6 days, or 7 days before the administration of the one or more immune checkpoint inhibitor. In some embodiments, the tubulin binding agent is administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 30 h, 36 h, 40 h, 48 h, 4 days, 5 days, 6 days, or 7 days before the administration of one or more immune checkpoint inhibitor. In some embodiments, the tubulin binding agent is administered in more than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 30 h, 36 h, 40 h, 48 h, 3 days, 4 days, 5 days, 6 days, or 7 days before the administration of the one or more immune checkpoint inhibitor.

In some embodiments, the treatment schedule includes co-administration of one or more immune checkpoint inhibitor and the tubulin binding agent. In some embodiments, the treatment schedule includes co-administration of one or more immune checkpoint inhibitor and the tubulin binding agent once every 1 week, 2 weeks, 3 weeks, 4 weeks, 5 weeks, 6 weeks, 7 weeks, or 8 weeks. In some embodiments, the treatment schedule includes co-administration of one or more immune checkpoint inhibitor and the tubulin binding agent two times every 1 week, 2 weeks, 3 weeks, 4 weeks, 5 weeks, 6 weeks, 7 weeks, or 8 weeks. In some embodiments, the treatment schedule includes co-administration of one or more immune checkpoint inhibitor and the tubulin binding agent once every 1 week in a treatment cycle of 1 week, 2 weeks, 3 weeks, 4 weeks, 5 weeks, 6 weeks, 7 weeks, or 8 weeks. In some embodiments, the treatment schedule includes co-administration of one or more immune checkpoint inhibitor and the tubulin binding agent twice every 1 week in a treatment cycle of 1 week, 2 weeks, 3 weeks, 4 weeks, 5 weeks, 6 weeks, 7 weeks, or 8 weeks. In some embodiments, the treatment schedule includes co-administration of one or more immune checkpoint inhibitor and the tubulin binding agent on day 1, day 8, and day 15 of a 21-day treatment cycle. In some embodiments, co-administration of one or more immune checkpoint inhibitor and the tubulin binding agent includes administering one or more immune checkpoint inhibitor prior to administering plinabulin. In some embodiments, co-administration of one or more immune checkpoint inhibitor and the tubulin binding agent includes administering one or more immune checkpoint inhibitor after administering plinabulin. In some embodiments, co-administration of one or more immune checkpoint inhibitor and the tubulin binding agent includes administering the one or more immune checkpoint inhibitor concurrently with the tubulin binding agent. In some embodiments, one or more immune checkpoint inhibitor described in this paragraph can independently be a first, second, third, fourth, fifth, sixth, seventh, or eighth immune checkpoint inhibitor. In some embodiments, the treatment schedule includes co-administration of one or more immune checkpoint inhibitor and v every day of the week for a week. In some embodiments, the treatment schedule includes co-administration of one or more immune checkpoint inhibitor and v every day of the week for 2 weeks, 3 weeks, or 4 weeks. In some embodiments, the treatment schedule includes co-administration of one or more immune checkpoint inhibitor and the tubulin binding agent on day 1 in weekly treatment. In some embodiments, the treatment schedule includes co-administration of one or more immune checkpoint inhibitor and the tubulin binding agent on day 1 and day 2 in weekly treatment. In some embodiments, the treatment schedule includes co-administration of one or more immune checkpoint inhibitor and the tubulin binding agent on day 1, day 2, and day 3 in weekly treatment. In some embodiments, the treatment schedule includes co-administration of one or more immune checkpoint inhibitor and the tubulin binding agent on day 1, day 2, day 3 in weekly treatment. In some embodiments, the treatment schedule includes co-administration of one or more immune checkpoint inhibitor and the tubulin binding agent on day 1, day 2, day 3, and day 4 in weekly treatment. In some embodiments, the treatment schedule includes co-administration of one or more immune checkpoint inhibitor and the tubulin binding agent on day 1, day 2, day 3, day 4, and day 5 in weekly treatment. In some embodiments, the treatment schedule includes co-administration of one or more immune checkpoint inhibitor and the tubulin binding agent on day 1, day 2, day 3, day 4, day 5, and day 6 in weekly treatment. In some embodiments, the treatment schedule includes co-administration of one or more immune checkpoint inhibitor composition and the tubulin binding agent on day 1, day 3, and day 5 in weekly treatment. In some embodiments, the treatment cycle for the tubulin binding agent and the one or more immune checkpoint inhibitors may be the same. In other embodiments, the treatment cycle for the tubulin binding agent and the one or more immune checkpoint inhibitors may be different. For example, in some embodiments, the treatment cycle for the tubulin binding agent is 21 days, whereas the treatment cycle for the one or more immune checkpoint inhibitors is 14 days. In some embodiments, one or more immune checkpoint inhibitor is used on each administration day can be the same or different. In some embodiments, one or more immune checkpoint inhibitor used on the first administration day is different from one or more immune checkpoint inhibitor used on the rest of the administration days. In some embodiments, one or more immune checkpoint inhibitor used on the first administration day is the same as or different from one or more immune checkpoint inhibitor used on the second administration day. In some embodiments, one or more immune checkpoint inhibitor used on the first administration day is the same as or different from one or more immune checkpoint inhibitor used on the third administration day. In some embodiments, one or more immune checkpoint inhibitor composition used on the first administration day is the same as or different from one or more immune checkpoint inhibitor used on the fourth administration day. In some embodiments, one or more immune checkpoint inhibitor used on the first administration day is the same as or different from one or more immune checkpoint inhibitor used on the fifth administration day. In some embodiments, one or more immune checkpoint inhibitor used on the first administration day is the same as or different from one or more immune checkpoint inhibitor used on the sixth administration day. In some embodiments, one or more immune checkpoint inhibitor used on the first administration day is the same as or different from one or more immune checkpoint inhibitor used on the seventh administration day.

In some embodiments, the treatment schedule includes administration of one or more immune checkpoint inhibitor (e.g., the first, the second, the third, the fourth, the fifth, the sixth, the seventh, or the eighth) once every 3 weeks. In some embodiments, the treatment schedule includes administration of one or more immune checkpoint inhibitor once every 1 week, 2 weeks, 3 weeks, 4 weeks, 5 weeks, 6 weeks, 7 weeks, or 8 weeks. In some embodiments, the treatment schedule includes administration of one or more immune checkpoint inhibitor two times every 1 week, 2 weeks, 3 weeks, 4 weeks, 5 weeks, 6 weeks, 7 weeks, or 8 weeks. In some embodiments, the treatment schedule includes administration of one or more immune checkpoint inhibitor once every 1 week in a treatment cycle of 1 week, 2 weeks, 3 weeks, 4 weeks, 5 weeks, 6 weeks, 7 weeks, or 8 weeks. In some embodiments, the treatment schedule includes administration of one or more immune checkpoint inhibitor twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of one or more immune checkpoint inhibitor three times (e.g., day 1, 2, 3, or day 1, 3, 5) every week in a treatment cycle of 1 week, 2 weeks, 3 weeks, 4 weeks, 5 weeks, 6 weeks, 7 weeks, or 8 weeks. In some embodiments, the treatment schedule includes administration of one or more immune checkpoint inhibitor day 1, day 8, and day 15 of a 21-day treatment cycle. The one or more immune checkpoint inhibitor described in this paragraph can independently be the first, second, third, fourth, fifth, sixth, seventh, or eighth one or more immune checkpoint inhibitor. In some embodiments, the treatment schedule includes administration of one or more immune checkpoint inhibitor every day of the week for a week. In some embodiments, the treatment schedule includes administration of the one or more immune checkpoint inhibitor every day of the week for 2 weeks, 3 weeks, or 4 weeks. In some embodiments, the treatment schedule includes administration of one or more immune checkpoint inhibitor composition on day 1 in weekly treatment. In some embodiments, the treatment schedule includes administration of one or more immune checkpoint inhibitor on day 1 and day 2 in weekly treatment. In some embodiments, the treatment schedule includes administration of one or more immune checkpoint inhibitor on day 1, day 2, and day 3 in weekly treatment. In some embodiments, the treatment schedule includes administration of one or more immune checkpoint inhibitor on day 1, day 3, day 5 in weekly treatment. In some embodiments, the treatment schedule includes administration of one or more immune checkpoint inhibitor on day 1, day 2, day 3, and day 4 in weekly treatment. In some embodiments, the treatment schedule includes administration of one or more immune checkpoint inhibitor on day 1, day 2, day 3, day 4, and day 5 in weekly treatment. In some embodiments, the treatment schedule includes administration of one or more immune checkpoint inhibitor on day 1, day 2, day 3, day 4, day 5, and day 6 in weekly treatment.

In some embodiments, the treatment schedule includes administration of v once every 3 weeks. In some embodiments, the treatment schedule includes administration of the tubulin binding agent once every 1 week, 2 weeks, 3 weeks, 4 weeks, 5 weeks, 6 weeks, 7 weeks, or 8 weeks. In some embodiments, the treatment schedule includes administration of the tubulin binding agent two times every 1 week, 2 weeks, 3 weeks, 4 weeks, 5 weeks, 6 weeks, 7 weeks, or 8 weeks. In some embodiments, the treatment schedule includes administration of the tubulin binding agent once every 1 week in a treatment cycle of 1 week, 2 weeks, 3 weeks, 4 weeks, 5 weeks, 6 weeks, 7 weeks, or 8 weeks. In some embodiments, the treatment schedule includes administration of the tubulin binding agent twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of the tubulin binding agent three times (e.g., day 1, 2, 3, or day 1, 3, 5) every 1 week in a treatment cycle of 1 week, 2 weeks, 3 weeks, 4 weeks, 5 weeks, 6 weeks, 7 weeks, or 8 weeks. In some embodiments, the treatment schedule includes administration of the tubulin binding agent on day 1 of a 21-day treatment cycle. In some embodiments, the treatment schedule includes administration of the tubulin binding agent on day 1 and day 8 of a 21-day treatment cycle. In some embodiments, the treatment schedule includes administration of the tubulin binding agent day 1, day 8, and day 15 of a 21-day treatment cycle. In some embodiments, the treatment schedule includes administration of the tubulin binding agent every day of the week for a week. In some embodiments, the treatment schedule includes administration of vevery day of the week for 2 weeks, 3 weeks, or 4 weeks. In some embodiments, the treatment schedule includes administration of the tubulin binding agent on day 1 in weekly treatment. In some embodiments, the treatment schedule includes administration of plinabulin on day 1 and day 2 in weekly treatment. In some embodiments, the treatment schedule includes administration of the tubulin binding agent on day 1, day 2, and day 3 in weekly treatment. In some embodiments, the treatment schedule includes administration of the tubulin binding agent on day 1, day 3, day 5 in weekly treatment. In some embodiments, the treatment schedule includes administration of the tubulin binding agent on day 1, day 2, day 3, and day 4 in weekly treatment. In some embodiments, the treatment schedule includes administration of the tubulin binding agent on day 1, day 2, day 3, day 4, and day 5 in weekly treatment. The treatment schedule includes administration of the tubulin binding agent on day 1, day 2, day 3, day 4, day 5, and day 6 in weekly treatment.

The treatment cycle can be repeated as long as the regimen is clinically tolerated. In some embodiments, the treatment cycle for one or more immune checkpoint inhibitor and vis repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a new treatment cycle can occur a period of time after the completion of the previous treatment cycle. In some embodiments, a new treatment cycle can occur after 1 week, 2 weeks, 3 weeks, 4 weeks, 5 weeks, 6 weeks, or 7 weeks after the completion of the previous treatment cycle.

Administration of the composition disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

In some embodiments, the compositions described herein can be used in combination with other therapeutic agents. In some embodiments, the compositions described herein can be administered or used in combination with treatments such as chemotherapy, radiation, and biologic therapies.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### EXAMPLES

Example 1

A single arm study utilizing patients with glioblastoma is conducted. The principle that will be employed is a triple combination therapy approach to (1) generate antigens (phospho-antigens) by inhibition of FPPS with zoledronic acid, (2) enhance antigen presentation by stimulating dendritic cells with plinabulin and (3) optimize CD8 T cell cytotoxic response with a PD-1/PD-L1 inhibitor.

A single arm study utilizing patients with glioblastoma is conducted. The principle that will be employed is a triple combination therapy approach to (1) generate antigens (phospho-antigens) by inhibition of FPPS with zoledronic acid, (2) enhance antigen presentation by stimulating dendritic cells with plinabulin and (3) optimize gamma-delta T cell cytotoxic response with a PD-1/PD-L1 inhibitor.

Patients will receive pretreatment with one or more doses of zoledronic acid to induce the generation of phosphoantigens. The patients will then receive plinabulin to present phosphoantigens to (1) gamma-delta T cells and (2) CD4/DC8 T-cells. The patients will then receive a PD-1 inhibitor or a PD-L 1 inhibitor. The patients will undergo surgical removal (debulking) of the glioblastoma tumor that will enable correlative analysis of the tissue.

The glioblastoma tissue will be analyzed for (1) FPPS quantitates, (2) phosphantigen quantities, (3) infiltrating T-cell repertoire (DC4, DC8, T-Regs, gamma-delta T-cells), and additional markers. The presence of phosphoantigens will be a positive indicator that the zoledonic acid penetrated the blood brain barrier.

Patients will be followed up for survival. The data will correlate FPPS expression, phosphoantigen production, T-cell repertoire in the glioblastoma tissue with duration of survival.

### Example 2

Synergy of plinabulin, an immune checkpoint inhibitor (PD-1 antibody) and a FPPS inhibitor (a nitro-bisphosphonate) is tested in comparison with the treatment with plinabulin alone, a PD-1 antibody alone, and a composition of plinabulin and a PD-1 antibody. The tests are performed using seven to ten-week old mice that are injected subcutaneously with glioblastoma multiforme tumor cells. Six testing groups are prepared, and each group includes nine mice.

Group 1 is administered with saline; Group 2 is administered with the plinabulin diluent (in the absence of plinabulin); Group 3 is administered with the plinabulin dissolved in diluent at a concentration of 7.5 mg/kg; Group 4 is administered with PD-1 antibody; Group 5 is administered with plinabulin/PD-1 antibody combined treatment; and Group 6 is administered with plinabulin/PD- 1 antibody/FPPS inhibitor combined treatment. For the plinabulin/PD-1 antibody/FPPS inhibitor combined treatment (Group 6), the mice are administered twice per week (Day 1 and Day 4 of each week) with Plinabulin (7.5 mg/kg) that is dissolved in diluent, followed by administering PD-1 antibody one hour after each plinabulin administration, followed by administering a FPPS inhibitor one hour after each PD-1 antibody administration. For the plinabulin only treatment (Group 3), the antibody only treatment (Group 4), or the plinabulin/PD-1 antibody treatment (Group 5) mice are administered Plinabulin (7.5 mg/kg dissolved in diluent) or antibody alone or in combination twice per week (Day 1 and Day 4 of each week). For Groups 1 and 2, the mice are administered with saline or the Plinabulin diluent alone twice per week.

Each treatment starts at tumor size of around 125 mm³ and continues until tumor size of 1500 mm³ is reached. If the mean tumor size in any group has not reached 1500 mm³ by Experimental Day 45, treatment will be stopped and tumor size continued to be assessed. To determine the efficacy of each treatment, the following data are collected: mortality rate prior to tumor size reaching 1500 mm³; the body weight of the mice assessed twice weekly both prior to treatments; the rate of tumor growth as determined by the tumor size measurement (twice every week); the tumor growth index; overall survival rate; and the time required to double tumor size.

What is claimed is:

1. A pharmaceutical composition, comprising a T-cell activator and/or proliferator, one or more immune checkpoint inhibitor, and a FPPS inhibitor.

2. The composition of claim 1, wherein the T-cell activator and/or proliferator is a tubulin binding agent.

3. The composition of claim 2, wherein the tubulin binding agent is selected from a group consisting of vinblastine, vincristine, vinorelbine, vinflunine, crytophycin 52, halichondrins, dolastatins, hemiasterlins, colchicine, combretastatins, 2-methyoxyestradiol, E7010, paclitaxel, docetaxel, epothilone, discodermolide, and plinabulin.

4. The composition of claim 2 or 3, wherein the tubulin binding agent is plinabulin.

5. The composition of claim 1, wherein the FPPS inhibitor is a nitrogen-containing bisphosphonate compound.

6. The composition of claim 1, wherein the FPPS inhibitor is quinolone derivative compound or an allosteric non-bisphosphonate compound.

7. The composition of claim 1 or 5, wherein the FPPS inhibitor is selected from pamidronate, alendronate, risedronate, zoledronate, and ibandronat, or an acid or salt thereof.

8. The composition of claim 1, wherein the immune checkpoint inhibitor is an inhibitor of PD-1, PD-L1, PD-L2, PD-L3, PD-L4, CTLA-4, LAGS, B7-H3, B7-H4, KIR or TIM3.

9. The composition of claim 1 or 8, wherein the immune checkpoint inhibitor is a PD-1 antibody, a PD-L 1 antibody, a PD-L2 antibody, a CTLA-4 antibody, or a combination thereof.

10. The composition of claim 9, wherein the a PD-1 antibody, a PD-L1 antibody, a PD-L2 antibody, a CTLA-4 antibody is selected from α-CD3-APC, α-CD3-APC-H7, α-CD4-ECD, α-CD4-PB, α-CD8-PE-Cy7, α-CD-8-PerCP-Cy5.5, α-CD 1 lc-APC, α-CD11b-PE-Cy7, α-CD11b-AF700, α-CD14-FITC, α-CD16-PB, α-CD19-AF780, α-CD19-AF700, α-CD2O-PO, α-CD25-PE-Cy7, α-CD40-APC, α-CD45-Biotin, Streptavidin-B V605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137.

11. The composition of any one of claims 1 to 10, further comprising one or more pharmaceutically acceptable excipients.

12. The composition of any one of claim 1 or 9, wherein the immune checkpoint inhibitor is nivolumab, pembrolizumab, pidilizumab, ipilimumab, BMS 936559, atezolizumab, durvalimumab, or any combinations thereof.

13. The composition of claim 1, further comprising one or more additional chemotherapeutic agent.

14. A method for treating or ameliorating cancer in a subject, comprising administering a T-cell activator, one or more immune checkpoint inhibitor, and a FPPS inhibitor to a subject in need thereof.

15. The method of claim 14, wherein the T-cell activator is a tubulin binding agent.

16. The method of claim 15, wherein the tubulin binding agent is selected from a group consisting of vinblastine, vincristine, vinorelbine, vinflunine, crytophycin 52, halichondrins, dolastatins, hemiasterlins, colchicine, combretastatins, 2-methyoxyestradiol, E7010, paclitaxel, docetaxel, epothilone, discodermolide, and plinabulin.

17. The method of claim 15 or 16, wherein the tubulin binding agent is plinabulin.

18. The method of claim 14, wherein the FPPS inhibitor is a nitrogen-containing bisphosphonate compound.

19. The method of claim 14, wherein the FPPS inhibitor is quinolone derivative compound or an allosteric non-bisphosphonate compound.

20. The method of claim 14 or 15, wherein the FPPS inhibitor is selected from pamidronate, alendronate, risedronate, zoledronate, and ibandronat, or an acid or salt thereof.

21. The method of claim 14, wherein the cancer comprises cells expressing farnesyl pyrophosphate synthetase.

22. The method of claim 14, wherein the cancer is a low-grade immunogenic cancer

23. The method of claim 14, wherein the cancer is rhabdoid tumor, Ewing sarcoma, thyroid cancer, acute myeloid leukemia (AML), medulloblastoma cancer, carcinoid cancer, neuroblastoma, prostate cancer, chronic lymphocytic leukemia (CLL), low-grade glioma, breast cancer, pancreas, multiple myeloma, kidney papillary cell, ovarian cancer, glioblastoma multiforme, cervical, diffuse large B-cell lymphoma (DLBCL), head and neck, colorectal, esophageal adenocarcinoma, bladder cancer, lung adenosacrinoma, lung squamous cell carcinoma, or melanoma.

24. The method of claim 14, wherein the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma.

25. The method of any one of claims 14 to 22, wherein the cancer is glioblastoma multiforme.

26. The method of any one of claims 14 to 25, wherein the immune checkpoint inhibitor is an inhibitor of PD-1, PD-L1, PD-L2, PD-L3, PD-L4, CTLA-4, LAG3, B7-H3, B7-H4, KIR or TIM3.

27. The method of claim 14, further comprising co-administering one or more additional chemotherapeutic agent.

28. The method of claim any one of claims 13 to 27, comprising co-administering a first immune checkpoint inhibitor and a second immune checkpoint inhibitor, wherein the first immune checkpoint inhibitor is different from the second immune checkpoint inhibitor.

29. The method of claim 28, wherein the first and the second immune checkpoint inhibitor is independently an inhibitor of PD-1, PD-L1, PD-L2, PD-L3, PD-L4, CTLA-4, LAG3, B7-H3, B7-H4, KIR or TIM3.

30. The method of claim 29, wherein the first immune checkpoint inhibitor is a PD-1 inhibitor, and the second immune checkpoint inhibitor is a CTLA-4 inhibitor.

31. The method of any one of claims 13 to 30, wherein the immune checkpoint inhibitor is an antibody.

32. The method of claim 31, wherein the immune checkpoint inhibitor is a PD-1 antibody, a PD-L1 antibody, a PD-L2 antibody, or a CTLA-4 antibody.

33. The method of claim 32, wherein the antibody is selected from α-CD3-APC, α-CD3-APC-H7, α-CD4-ECD, α-CD4-PB, α-CD8-PE-Cy7, α-CD-8-PerCP-Cy5.5, α-CD11c-APC, α-CD11b-PE-Cy7, α-CD11b-AF700, α-CD14-FITC, α-CD16-PB, α-CD19-AF780, α-CD19-AF700, α-CD2O-PO, α-CD25-PE-Cy7, α-CD40-APC, α-CD45-Biotin, Streptavidin-BV605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-Pα, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137.
